(19) United States
(12) Patent Application Publication
(10) Pub. No.: US 2021/0196858 A1
(43) Pub. Date: Jul. 1, 2021

(54) SHAPEABLE DEMINERALIZED BONE MATRIX PRODUCTS AND METHOD OF MANUFACTURE THEREOF

(71) Applicants: Helena Lovick, N. Great Falls, MT (US); Ross Wolfe, Belgrade, MT (US)

(72) Inventors: Helena Lovick, N. Great Falls, MT (US); Ross Wolfe, Belgrade, MT (US)

(21) Appl. No.: 17/138,284

(22) Filed: Dec. 30, 2020

Related U.S. Application Data
(60) Provisional application No. 62/955,896, filed on Dec. 31, 2019.

(51) Int. Cl. A61L 27/36; A61F 2/30

(52) U.S. Cl. CPC A61L 27/3608; A61F 2/30965; A61L 27/3691; A61L 27/365; A61F 2002/2835; A61F 2002/30263; A61F 2002/3093; A61F 2002/30069; A61F 2002/30224

(57) ABSTRACT

The present invention relates to pliable, compression-resistant bone-based products and methods of making the same.

### CROSS REFERENCE TO RELATED APPLICATIONS

This application claims priority and the benefit under 35 U.S.C. § 119(e) to U.S. Provisional Patent Application Ser. No. 62/955,896, filed Dec. 31, 2019. This reference is incorporated herein in its entirety by reference.

### FIELD OF THE INVENTION

The present embodiments generally relate to pliable, compression-resistant bone-based products and a method of manufacturing thereof.

### BACKGROUND

Methods for manufacturing shaped, bone-based products are known in the art. In general, these methods require the use of excipients, carriers and/or specialized processing conditions (pressure, lyophilization, etc.) to generate products of the desired shape and physical properties.

U.S. Pat. No. 7,045,514 to Merboth et al. (incorporated in its entirety by reference) discloses a bone repair composition comprising a mixture of bone material with a gelatin- and hydrogel-based carrier. The bone material comprises between about 20-65 wt. % of the composition and the gelatin component ranges from about 10-50 wt. % of the composition. Merboth describes a method of production of the bone composition where the composition is flattened, cooled to room temperature and cut into sheets for use.

U.S. Pat. No. 7,785,634 to Borden (incorporated in its entirety by reference) discloses the production of a bone product comprising a mineralized carrier and at least one bone graft material. The mineralized carrier comprises solubilized minerals from bone obtained directly from cortical bone, not demineralized bone matrix, via extraction of a gelatin solution from bone with acid. The extraction process involves heating of the bone in the presence of an acidic extraction solution, sonication, and isolation of the extraction solution from the residual bone. The extraction can be repeated with fresh acid and the extraction solution lyophilized to provide “freeze dried mineralized gelatin”. After isolation of the freeze-dried gelatin, it can be combined with water and demineralized bone matrix (DBM) to form a DBM putty.

U.S. Pat. No. 6,576,249 to Gendler et al. (incorporated in its entirety by reference) discloses a method for preparation of bone gel and bone putty comprising dissolving demineralized bone matrix in water or an aqueous solution at temperatures above 25° C. The resultant supernatant, with a viscosity above 2 centipoise, is cooled and mixed with bone particles to form a gel-like suspension or putty-like material. The art of making gelatin has been known for centuries on the basis of its food applications. In order to produce bone-derived gelatin with superior properties for bone grafting applications, the method of manufacture requires modification.

### SUMMARY OF THE INVENTION

The present invention discloses products that are advantageous over this prior art. The disclosed invention is directed to a compression-resistant bone-based product and a method of manufacturing thereof. The bone-based product can be shaped into a three-dimensional form of dimensions established by the use of a mold and drying conditions. The properties of the compression-resistant bone-based product demonstrate increased compression resistance over products manufactured in the absence of selected additives. The method of manufacturing relies on the judicious selection of an aqueous carrier, bone particle sizes, and processing pressure and temperature conditions. In some embodiments, the method of manufacture also includes the use of a mold and drying conditions.

The bone can be cortical bone, cancellous bone, or combinations thereof. In some embodiments, the bone can be fully demineralized, partially demineralized, mineralized or any combinations thereof. The bone-based products can be partially dehydrated, fully dehydrated, or fully hydrated. The bone can be allogeneic, autogeneic, and xenogeneic tissues, and combinations thereof.

When hydrated (greater than about 30% to about 80% water content), the bone-based articles of the invention compress under a force of between about 5 g-force/square cm to about 4000 g-force/square cm. The hydrated bone-based products can be compressible to between about 95% and about 70% of its original size, in some embodiments about 80% of its original size. When dehydrated to less than about 10% residual moisture, the bone-based articles of the invention demonstrate a compressive load of about 60 lbf to about 15 lbf for a cube of about 0.20 cm³ in a double lap shear test.

The product can be in the form of a putty, paste, or rigid solid. The residual moisture content of the rigid solid form of the product can be less than about 10%. In some embodiments, the residual moisture content of the rigid solid form of the product can be about 0%, about 1%, about 2%, about 3%, about 4%, about 5%, about 6%, about 7%, about 8%, about 9%, or about 10%. The bone-based product can be osteoinductive.

An aspect of the invention is a three-dimensional compression-resistant, bone-based product of specific dimensions. In some embodiments, the article can be shaped in a mold prior to a drying step. In other embodiments, after drying, the shape can be milled or cut down to form an alternative shape.

An aspect of the invention is a method for forming a compression-resistant, bone-based product. The bone used in the manufacture of the bone-based article can be in a variety of forms such as powder, chips, fibers, or combinations thereof. The bone can be mineralized, demineralized, partially demineralized, or a combination thereof. In some embodiments, other biocompatible components can be added to the bone-based product during manufacture.

After sizing of the bone pieces, the bone is combined with a treatment solution. The treatment solution can be aqueous solutions including, but not limited to, an aqueous acid, an aqueous base, a balanced salt solution, a buffer, Hank's balanced salt solution, phosphate buffered saline, phosphate solutions, saline or combinations thereof. After combining with a treatment solution, the bone and aqueous solution mixture can be softened. After softening of the bone pieces, the mixture can be combined with additional bone pieces or other biocompatible components.

In some embodiments, the manufacturing process can include a drying or dehydration step such as lyophilization. In some embodiments, the drying or dehydration step can include holding the bone mixture in a mold at temperatures below about 0° C. (in some embodiments between about 0° C. and about −80° C.) under reduced pressure of at least about half of atmospheric pressure, less than about 380 mm Hg, in some embodiments between about 0.1 mm Hg and about 10 mm Hg.

### BRIEF DESCRIPTION OF THE FIGURES

FIG. 1 illustrates a process flowchart for the production of pliable, compression-resistant bone-based products manufactured by the method of the present invention.

### DETAILED DESCRIPTION OF THE INVENTION

The present invention relates to a shapeable, pliable, compression-resistant bone-based article and methods of making the same.

“Allogeneic” or “allograft”, as used herein, refers to tissue derived from a non-identical donor of the same species, which may be a DBM.

“Atmospheric pressure”, as used herein, refers to the pressure exerted by the weight of earth's atmosphere at the location of manufacturing.

“Autogeneic” or “autograft”, as used herein, refers to tissue derived from and implanted into the same identical patient.

“Biocompatible”, as used herein, refers to the property of being biologically compatible with a living being by not causing harm.

“Fully demineralized”, as used herein, is refers to bone with less than about 8% residual calcium content. “Osteoinductive”, as used herein, refers to the ability of a material to induce bone healing via recruitment of osteoprogenitor cells.

“Partially demineralized”, as used herein, is refers to bone with more than 8% residual calcium content but less calcium content than the native, unprocessed mineralized bone tissue.

“Patient”, as used herein, refers to a living recipient of the biomaterial-based implants of the present invention.

“Room temperature”, as used herein, is defined as temperatures ranging from about 18 to about 27 degrees Celsius.

“Xenogeneic” or “xenograft”, as used herein, is defined as tissue derived from a non-identical donor of a different species.

The shapeable, pliable, compression-resistant bone-based article of the invention have many advantages over the prior art. When hydrated (greater than about 30% to about 80% water content), the bone-based articles of the invention can compress under a force of between about 5 g-force/square cm to about 4000 g-force/square cm. The hydrated bone-based articles can be compressible to about 80% of its original size, to about 60% of its original size, to about 20% of its original size, to about 5% of its original size, or any range defined by two or more values, or any value or range within the greatest range, without loss of structural integrity or bone cohesion (e.g. the cohesion is 200 kN/m²). When dehydrated to less than about 10% residual moisture, the bone-based articles of the invention demonstrate a compressive load of about 60 lbf to about 15 lbf for a cube of about 0.20 cm³ in a double lap shear test.

The shapeable article can be in the form of a putty, paste, or rigid solid. The bone-based article can be composed of a single material or a mixture of materials, which can be used as scaffolding during bone regrowth. Suitable aqueous fluids for inclusion within the bone-based article include, but are not limited to, water, saline, buffer, balanced salt solution, blood, bone marrow aspirate, plasma and combinations thereof. In some embodiments, the bone-based product can be composed solely of bone tissue. Furthermore, while the invention may be used to produce a shape that may later be milled or cut down to form an alternative shape, the invention allows for the shape to be formed without this additional step of cutting. In some embodiments, the bone-based article can be shaped in a mold prior to a drying step. The bone-based material of the invention can be in the form of fibers, powder, or chips. In some embodiments, the bone-based material can be sized to a selected size distribution, e.g., about 200 μm to about 4 mm. In some embodiments, the bone pieces are sized into particles of about 100 microns to about 4 mm in size, to about 1 mm to about 4 mm, to about 200 microns to about 1 mm, to about 200 microns to about 850 microns. In some embodiments, the bone pieces are sized to about 100 microns, about 200 microns, about 250 microns, about 300 microns, about 350 microns, about 400 microns, about 450 microns, about 500 microns, about 550 microns, about 600 microns, about 650 microns, about 700 microns, about 750 microns, about 800 microns or about 850 microns, or a range defined by two of these values, or a value or range within the greatest range. In other embodiments, the bone can be sized into multiple, different sizes. For example, one-third of the bone material can be sized to about 1 mm to about 4 mm and the remaining two-thirds of the bone material can be sized to about 200 microns to about 850 microns. Bone fibers for use in the bone-based product can be of lengths of about 1 mm to about 200 mm, of about 2 mm to about 150 mm, of about 5 mm to about 70 mm, to about 10 mm to about 60 mm. In some embodiments, the length of the bone fibers can be about 1 mm, about 5 mm, about 10 mm, about 15 mm, about 20 mm, about 25 mm, about 30 mm, about 35 mm, about 40 mm, about 45 mm, about 50 mm, 60 mm, about 70 mm, about 80 mm, about 90 mm, about 100 mm, about 110 mm, about 120 mm, about 130 mm, about 140 mm, about 150 mm, about 160 mm, about 170 mm, about 180 mm, about 190 mm, or about 200 mm, or any range defined by two values set forth, or a value or range set forth within the largest range. The average length of the fibers can be between about 15 mm to about 50 mm, in some embodiments about 30 mm. The fibers can have a width or diameter of about 0.1 mm to about 30 mm, of about 0.2 mm to about 15 mm, of about 0.5 mm to about 10 mm, to about 1 mm to about 8 mm. In some embodiments, the width of the bone fibers can be about 0.1 mm, about 0.5 mm, about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 8 mm, about 9 mm, about 10 mm, about 15 mm, about 20 mm, about 25 mm, or about 30 mm, or any range defined by two values set forth, or a value or range set forth within the largest range. The average width or diameter can be between about 1 mm to about 5 mm, in some embodiments between about 2-3 mm.

The bone-based product can have about 30% to 80% moisture content. When the product is a putty or paste, and has about 30% to about 80% moisture content, the viscosity at room temperatures ranges from about 30,000 centipoise to about 300,000 centipoise depending on the selected method of manufacture. The inclusion of other additives and bone materials into the bone-based product increases the viscosity of the product. In some embodiments, the bone-based product can have less than about 10% residual moisture.

The bone-based product can be implanted in the hydrated form (about 30% to about 80% moisture content). In other embodiments, the bone-based product can be implanted in the dehydrated form with less than about 10% residual moisture. Utilizing lyophilization parameters of less than about half of atmospheric pressure (760 mm Hg) and temperatures of less than about 0° C., the bone-based product can be dried to less than about 10% residual moisture.

In some embodiments, the bone-based product can be dried in a mold. The mold can be in the shape of a cube, a block, a strip, a cylinder, and a sphere. In some embodiments the molds can contain holes or an opening on one side to permit the removal of water during drying. The mold can be made from ceramic, aluminum, stainless steel, other metals, and combinations thereof. The mold can have at least one opening that can be a gap, a perforation, a screen, a slit, a shape and combinations of openings. The mold can be capable of withstanding steam sterilization. The mold can have variable dimensions, which can be determined by an assessment of the void to be filled in the patient. The mold can include a lid, which can be attached to the mold or detached from the mold. The final shape of the product is within about 10% of its projected size based on its pre-mold size after lyophilization (less than about 380 mm Hg and less than about 0° C.).

In some embodiments, other biocompatible components can be added to the bone-based product during processing. Suitable additives include, but are not limited to, preserving agents (e.g., cryopreservatives), bioactive agents, growth factors, hormones, cells, antibiotics, biocompatible minerals, antimicrobials, or combinations thereof. The mass component of additives to bone-based product may range from about 1% to about 80%, about 5% to about 50%. In some embodiments, the mass component of additive to the bone-based product can be about 1%, about 2%, about 5%, about 10%, about 15%, about 20%, about 25%, about 30%, about 40%, about 45%, about 50%, about 55%, about 60%, about 65%, about 70%, about 75%, about 80%, or any value or range between two of these values. Suitable antimicrobial agents include, but are not limited to, bisbiguanides, silver nanoparticles, silver nitrate, silver oxide, silver salts, silver sulfadiazine, silver zeolites, triclosan, antifolates, aminoglycosides, carbapenems, cephalosporins, fluoroquinolines, glycopeptides, macrolides, monobactams, oxazolidones, penicillins, rifamycins, sulfonamides and tetracyclines. The mass ranges of antimicrobial agents to bone-based product may range from about 1% to about 80%, about 5% to about 50%. The mass component of the antimicrobial agent to the bone-based product can be about 1%, about 2%, about 5%, about 10%, about 15%, about 20%, about 25%, about 30%, about 40%, about 45%, about 50%, about 55%, about 60%, about 65%, about 70%, about 75%, about 80%, or any value or range between two of these values. The foregoing additives can be used individually or as a mixture of multiple agents.

In some embodiments, the bone-based article can be placed within an external implant or cage. The external implant or cage can be a medical device. The external implant or cage can be composed of biocompatible materials. The products can be formed into shapes including, but not limited to, cylinders, cubes, blocks, strips, spheres, discs, and doughnuts. In some embodiments, the product can be shaped specifically to fill a bone void. The void can be determined by pre-assessment of a void, such as a bone void within a patient. The final use of the product can be placement within a void of a patient.

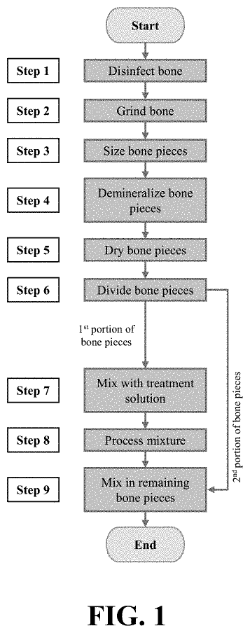

FIG. 1 illustrates a flowchart process map for the production of the bone-based articles in an aspect of the invention. For Step 1, the base material, bone, can be disinfected by a variety of means known in the art. These include, alcohol soaks, hydrogen peroxide soaks, coating with topic antiseptics, and so forth. The bone tissues can be allogeneic, autogeneic, or xenogeneic. The bone material can be cortical bone, cancellous bone or combination thereof. After disinfection, the bone is sized into smaller pieces (Step 2). This size reduction can be performed by milling, grinding, sawing, or other suitable method known in the art. In some embodiments, the bone is sized to a range of about 100 microns to about 4 mm in size, to about 1 mm to about 4 mm, to about 200 microns to about 1 mm, to about 200 microns to about 850 microns. Bone fibers for use in the bone-based product can be of lengths of about 1 mm to about 200 mm, of about 2 mm to about 150 mm, of about 5 mm to about 70 mm, to about 10 mm to about 60 mm. The average length of the fibers can be between about 15 mm to about 50 mm, in some embodiments about 30 mm. The fibers can have a width or diameter of about 0.1 mm to about 30 mm, of about 0.2 mm to about 15 mm, of about 0.5 mm to about 10 mm, to about 1 mm to about 8 mm. The average width or diameter can be between about 1 mm to about 5 mm, in some embodiments between about 2-3 mm. In other embodiments, the bone can be sized into a multiple, different sizes and shapes. For example, one-third of the bone material can be sized to about 1 mm to about 4 mm and the remaining two-thirds of the bone material can be sized to about 200 microns to about 850 microns. In another example, one-third of the bone can be sized to about 200 microns to about 850 microns and the remaining two-thirds of the bone material can be sized into fibers of about 1 mm diameter and about 150 mm long. For demineralization (Step 3), the bone can be prepared using any method or techniques known in the art, for a typical demineralization protocol, for example U.S. Pat. No. 5,314,476, or U.S. Pat. No. 8,574,825, each of which is incorporated in their entirety by reference. In some embodiments, Step 3 can be omitted or partially omitted, as the bone can be mineralized, fully demineralized, partially demineralized, or a combination of the foregoing.

After Step 3 or after Step 4 (if demineralization takes place), the bone pieces are dried to less than about 10% residual moisture. This dehydration can be via air-drying, oven-drying, and freeze-drying, combinations of the foregoing, or other suitable methods known in the art. After drying, the material can be divided into two groups of bone pieces. The two groups can be of bone pieces sized to the same size range from Step 2, or the groups can have different bone piece size distributions within the bone piece group and/or from the other bone piece group. Step 6 may be omitted and all sized bone pieces may go into Step 7 together. During Step 7, the bone pieces are mixed with a treatment solution. The treatment solution can be aqueous solutions, including, but not limited to, an aqueous acid, an aqueous base, a balanced salt solution, a buffer, Hank's balanced salt solution, phosphate buffered saline, phosphate solutions, saline or combinations thereof. In some embodiments, the treatment solution can include additional additives, such as calcium silicate, calcium chloride, and so forth. The bone pieces (grams) can be mixed with the aqueous solution (mL) in a ratio of about 1:1, about 3:7, about 2:8, about 1:9, about 1:19, with preferably more aqueous solution than bone pieces by weight. After the bone pieces are added to the aqueous solution, the mixture is stirred, vortexed, shaken, or agitated by mechanical means to produce a visually uniform mixture. During Step 8, the mixture is treated to soften the bone pieces. There are various techniques that can be used to soften the bone pieces.

Step 8. Via Extended Soak Times

The softening of bone pieces can take place by soaking the mixture within the aqueous solution for about 24 hours to about 48 hours. The soaking time can be about 24 hours, about 30 hours, about 35 hours, about 40 hours, or about 48 hours, or any range defined by two values set forth, or a value or range set forth within the largest range. In some embodiments, the mixture is soaked at temperatures of about 2° C. to about 8° C. In some embodiments, the temperature can be about 2° C., about 3° C., about 4° C., about 5° C., about 6° C., about 7° C., or about 8° C., or any range defined by two values set forth, or a value or range set forth within the largest range.

Step 8. Via Heating at Atmospheric Pressure

The softening of bone pieces can take place by heating the mixture at temperatures greater than about 60° C. for less than 12 hours. In some embodiments, the temperature can be between about 70° C. to about 90° C. and about 100° C. In some embodiments, the temperature can be about 60° C., about 65° C., about 70° C., about 75° C., about 80° C., about 85° C., about 90° C., about 95° C., or about 100° C., or any range defined by two values set forth, or a value or range set forth within the largest range. In some embodiments, the time of heating can be about 2 hours to about 8 hours, about 1 hour to about 4 hours. In some embodiments, the time can be about 2 hours, about 3 hours, about 4 hours, about 5 hours, about 6 hours, about 7 hours, or about 8 hours, or any range defined by two values set forth, or a value or range set forth within the largest range.

Step 8. Via Heating at Elevated Pressure

The softening of the bone pieces can take place by heating the mixture at temperatures greater than about 100° C. at pressures greater than about 14.7 psi. In some embodiments, the pressure can be about 14.75 psi to about 16.0 psi. In some embodiments the temperature can be about 100° C. to about 125° C. In some embodiments, the temperature can be about 100° C., about 105° C., about 110° C., about 115° C., about 120° C., or about 125° C., or any range defined by two values set forth, or a value or range set forth within the largest range. In other embodiments, the pressure is about 15 psi.

After the completion of the mixture processing (Step 8), the mixture can be mixed with the remaining bone pieces (Step 9). If heated, the mixture can be cooled to room temperature prior to mixing with additional components. In further embodiments, the treated bone mixture can be combined with other additives in addition to any remaining bone pieces or in the absence of any remaining bone pieces. Suitable additives include, but are not limited to, preserving agents (e.g., cryopreservatives), bioactive agents, growth factors, hormones, cells, antibiotics, biocompatible minerals, antimicrobials, or combinations thereof. Bioactive agents include, but are not limited to, bioactive glass composites, calcium phosphates, biphasic calcium phosphates, calcium silicates, hydroxyapatites, and combinations thereof. Growth factors and hormones can include, but are not limited to, calcium regulating hormones, parathyroid hormone (PHT), transforming growth factor beta (TGF-beta), prostaglandin E2 (PGE2), bone morphogenetic proteins (BMPs, e.g., BMP-2), insulin like growth factors (IGFs, e.g., IGF-1), and combinations thereof. The growth factors and hormones are native to the bone tissue. The cells can include mesenchymal stem cells, blood cells, osteogenic cell lines, chondrogenic cell lines, and combinations thereof. Suitable antimicrobial agents include, but are not limited to, bisbiguanides, silver nanoparticles, silver nitrate, silver oxide, silver salts, silver sulfadiazine, silver zeolites, triclosan, antifolates, aminoglycosides, carbapenems, cephalosporins, fluoroquinolines, glycopeptides, macrolides, monobactams, oxazolidones, penicillins, rifamycins, sulfonamides and tetracyclines. The foregoing additives can be used individually or as a mixture of multiple agents. The mass ratio of additives to bone material (grams) can range from about 1:2 to about 1:100. In some embodiments, the ratio of additives to bone material can be about 1:2, about 1:10, about 1:20, about 1:30, about 1:40, about 1:50, about 1:60, about 1:70, about 1:80, about 1:90, or about 1:100, or any range defined by two values set forth, or a value or range set forth within the largest range.

The products can be combined with a biologic material prior to, or during use. Suitable biologic materials include, but are not limited to, bone marrow aspirate, whole blood, plasma, serum albumin, concentrated bone marrow aspirate, platelet rich plasma, mesenchymal stem cells, osteogenic cell lines, chondrogenic cell lines, connective tissue progenitor cells, and combinations thereof.

The process illustrated in FIG. 1 can be further modified by lyophilizing the resultant mixture of Step 9. In some embodiments, after completion of Step 9, the mixture can be lyophilized to a residual moisture content of less than about 6%. The final article following lyophilization at this stage can be used as an implant without rehydration.

### EXAMPLES

### Example 1

Demineralized cortical bone powder of sized to approximately 200-900 μm was combined with water at a concentration of 25% w/w. The slurry was treated at 120° C./15 psi for 15 minutes. After the solution cooled to room temperature, the resultant gel was weighed. Additional demineralized bone powder sized to approximately 200-900 μm was stirred into the gel at a concentration of 15% w/w. The mixture was then poured into a mold and stored at −80° C. until lyophilization. The shaped material was lyophilized to a residual moisture content of about 6%.

Block samples of the lyophilized composition in the dimensions of 12.7 mm by 5.3 mm by 3.1 mm were subjected to a double lap shear compression test using an Instron 3343. With a load target of 118 pounds-force (lbf) at a rate of 0.200 inch per minute. The measured compressive load of the samples were 18.7 lbf.

Ranges have been discussed and used within the forgoing description. One skilled in the art would understand that any sub-range within the stated range would be suitable, as would any number within the broad range, without deviating from the invention.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

1. A bone-based product of specific dimensions comprising:
an aqueous carrier;
a plurality of bone pieces, wherein an average dimension of the plurality of bone pieces is between about 100 μm to about 2 mm, and wherein the product exhibits retained cohesion and compression resistance.

2. The article of claim 1, wherein a shape is selected from the group consisting of a cube, a block, a strip, cylinder, and a sphere.

3. The article of claim 1, wherein the plurality of bone pieces is selected from the group consisting of cortical bone, cancellous bone, and combinations thereof.

4. The article of claim 1, wherein the plurality of bone pieces is at least one of a fully demineralized bone, a partially demineralized bone and a mineralized bone.

5. The article of claim 1, wherein the plurality of bone pieces is at least one of a partially dehydrated, a fully dehydrated, or a fully hydrated.

6. The article of claim 1, wherein the plurality of bone pieces is selected from the group comprising allogeneic, autogeneic, and xenogeneic tissues, and combinations thereof.

7. The article of claim 1, wherein a moisture content of the product is between about 0% and 85%.

8. The article of claim 7, wherein a moisture content of the product is less than 6%.

9. The article of claim 1, wherein the product is osteoinductive.

10. A method of forming a bone-based product with retained exhibits retained cohesion, compression resistance, and flexibility comprising:
cutting bone into pieces;
placing a portion of bone pieces in an aqueous solution to form a mixture;
heating the mixture at elevated pressure to produce a heated mixture;
cooling the heated mixture to a produce a temperature and pressure-treated composition.

11. The method of claim 10, further comprising shaping the cooled temperature- and pressure-treated composition to a desired shape.

12. The method of claim 10, further comprising adding at least one additive to the temperature- and pressure-treated composition after heating the mixture at elevated pressure.

13. The method of claim 12, wherein the additives are additional bone pieces.

14. The method of claim 13, wherein the bone pieces are selected from the group consisting of cortical bone, cancellous bone, and combinations thereof.

15. The method of claim 13, wherein the bone pieces are at least one of a demineralized, a partially demineralized, a mineralized, or a combination of the foregoing.

16. The method of claim 10, wherein the bone is at least one of a demineralized bone, a partially demineralized bone, or a mineralized bone.

17. The method of claim 10, wherein the heating step heats the mixture to a temperature between about 80° C. and about 130° C.

18. The method of claim 10, wherein the heating step is performed at atmospheric pressure.

19. The method of claim 10, wherein the heating step is heating in a mold under elevated pressure of between about 14.7 psi to about 30 psi.
